(19) United States
(12) Patent Application Publication
(10) Pub. No.: US 2025/0307642 A1
(43) Pub. Date: Oct. 2, 2025

(54) TRAINING ML MODELS VIA REINFORCEMENT LEARNING FROM HUMAN FEEDBACK (RLHF) USING EMOTION DETECTION

(71) Applicant: Sony Interactive Entertainment Inc., Tokyo (JP)

(72) Inventor: Warren Benedetto, San Mateo, CA (US)

(21) Appl. No.: 18/623,785

(22) Filed: Apr. 1, 2024

(51) Int. Cl. G06N 3/092; A61B 5/16; G06N 3/0475

(52) U.S. Cl. CPC G06N 3/092; A61B 5/165; G06N 3/0475

(57) ABSTRACT

To reduce unconscious or unintended bias in evaluating the output of a machine learning (ML) model using reinforcement learning from human feedback, the emotions of a test human evaluating the model output are used in addition to or in lieu of evaluation input to train the model. As an example, if the sensed emotions do not match the evaluation input, the evaluation input may be discounted including discarding it altogether.

### FIELD

The present application relates generally to training machine learning (ML) models via reinforcement learning from human feedback (RLHF) using emotion detection.

### BACKGROUND

The process of reinforcement learning for a ML model for applications such as text, image, and video generation trains the model based on reward maximization. Using reward maximization, the model learns to make decisions that will generate the highest rewards. By incorporating human feedback into the reward function, ML models can be trained to make decisions that are better aligned with human preferences, needs, desires, and values. Human feedback, referred to herein as reinforcement learning from human feedback (RLHF) is an important factor in how large language models (LLM) can generate consistently believable text output, or how diffusion-based image models can generate realistic images.

### SUMMARY

As understood herein, the RLHF process generally involves presenting a human with responses from an ML model and asks the human to score which response sounds more human. The human may be asked to assess subjective characteristics which are difficult for a machine to evaluate, such as tone of voice, mood, context, etc. These responses are used to build the reward model, which is then used to optimize the language model. For example, the human may evaluate the model output and input a score into the feedback system. Or, the human may choose between two images based on a subjective evaluation of which is preferable.

As further understood herein, RLHF may at times be misleading because the human can introduce an element of bias into the evaluation. For example, a human may score the output based on how the person believes he or she “should” score it instead of how they actually feel. As examples, the human may think something is funny but fear that most people would find it offensive, so the human may reduce the score. Or, the human may think a version of text is accurate but that its content is objectionable and thus give it a lower score. Yet again, the human may feel a certain way about the output content from the model but believe his or her employer would disagree with the person's sentiment, and so score the output accordingly. Furthermore, a human may have an instinctual first reaction to the content, but upon reflection, may seek to justify or discount the initial response in favor of something the person have been able to rationalize with further thought. This risks introducing cognitive biases into the reward function.

Accordingly, a method includes presenting at least a first output from at least one machine learning (ML) model on at least one display and/or at least one speaker. The method also includes receiving a selection with respect to the output, identifying at least one emotion of a person making the selection, and responsive to the emotion satisfying an inconsistency threshold with respect to the selection, discounting the selection in training the ML model.

In some embodiments discounting the selection can include discarding the selection from training the ML model. In other embodiments discounting the selection can include according a first weight to the selection in training the ML model, with the first weight being less than a second weight of a non-discounted selection. Determining whether the emotion satisfies the inconsistency threshold may be done by comparing the emotion to the selection.

Without limitation, the selection may be received via a point-and-click device or a microphone. In some implementations the output of the ML model includes at least one image. In other implementations the output includes text.

In another aspect, a processor system is configured to associate at least one emotion signal indicating an emotion of a person with at least one evaluation input indicating an evaluation of the person of at least one audio and/or video output of at least one machine learning (ML) model trained to generate text and/or images. The processor system is configured to execute reinforcement learning from human feedback (RLHF) on the ML model according to the association of the emotion signal with the evaluation input.

In another aspect, a device includes a computer memory that is not a transitory signal and that in turn includes instructions executable by at least one processor system for, presenting output from a machine learning (ML) model, receiving at least one emotion signal from at least one person, and correlating the emotion signal with the output. The instructions are executable for executing reinforcement learning on the ML model according to the correlating of the emotion signal with the output.

The details of the present application, both as to its structure and operation, can be best understood in reference to the accompanying drawings, in which like reference numerals refer to like parts, and in which:

### BRIEF DESCRIPTION OF THE DRAWINGS

FIG. 1 is a block diagram of an example system in accordance with present principles;

FIG. 2 illustrates an example reinforcement learning system consistent with present principles;

FIG. 3 illustrates example overall logic in example flow chart format consistent with present principles;

FIG. 4 illustrates example specific logic in example flow chart format consistent with FIG. 3;

FIG. 5 illustrates an example screen shot of reinforcement learning consistent with present principles; and

FIG. 6 illustrates example alternative logic in example flow chart format consistent with present principles.

### DETAILED DESCRIPTION

This disclosure relates generally to computer ecosystems including aspects of consumer electronics (CE) device networks such as but not limited to computer game networks. A system herein may include server and client components which may be connected over a network such that data may be exchanged between the client and server components. The client components may include one or more computing devices including game consoles such as Sony PlayStation® or a game console made by Microsoft or Nintendo or other manufacturer, extended reality (XR) headsets such as virtual reality (VR) headsets, augmented reality (AR) headsets, portable televisions (e.g., smart TVs, Internet-enabled TVs), portable computers such as laptops and tablet computers, and other mobile devices including smart phones and additional examples discussed below. These client devices may operate with a variety of operating environments. For example, some of the client computers may employ, as examples, Linux operating systems, operating systems from Microsoft, or a Unix operating system, or operating systems produced by Apple, Inc., or Google, or a Berkeley Software Distribution or Berkeley Standard Distribution (BSD) OS including descendants of BSD. These operating environments may be used to execute one or more browsing programs, such as a browser made by Microsoft or Google or Mozilla or other browser program that can access websites hosted by the Internet servers discussed below. Also, an operating environment according to present principles may be used to execute one or more computer game programs.

Servers and/or gateways may be used that may include one or more processors executing instructions that configure the servers to receive and transmit data over a network such as the Internet. Or a client and server can be connected over a local intranet or a virtual private network. A server or controller may be instantiated by a game console such as a Sony PlayStation®, a personal computer, etc.

Information may be exchanged over a network between the clients and servers. To this end and for security, servers and/or clients can include firewalls, load balancers, temporary storages, and proxies, and other network infrastructure for reliability and security. One or more servers may form an apparatus that implement methods of providing a secure community such as an online social website or gamer network to network members.

A processor may be a single- or multi-chip processor that can execute logic by means of various lines such as address lines, data lines, and control lines and registers and shift registers. A processor including a digital signal processor (DSP) may be an embodiment of circuitry. A processor system may include one or more processors.

Components included in one embodiment can be used in other embodiments in any appropriate combination. For example, any of the various components described herein and/or depicted in the Figures may be combined, interchanged, or excluded from other embodiments.

“A system having at least one of A, B, and C” (likewise “a system having at least one of A, B, or C” and “a system having at least one of A, B, C”) includes systems that have A alone, B alone, C alone, A and B together, A and C together, B and C together, and/or A, B, and C together.

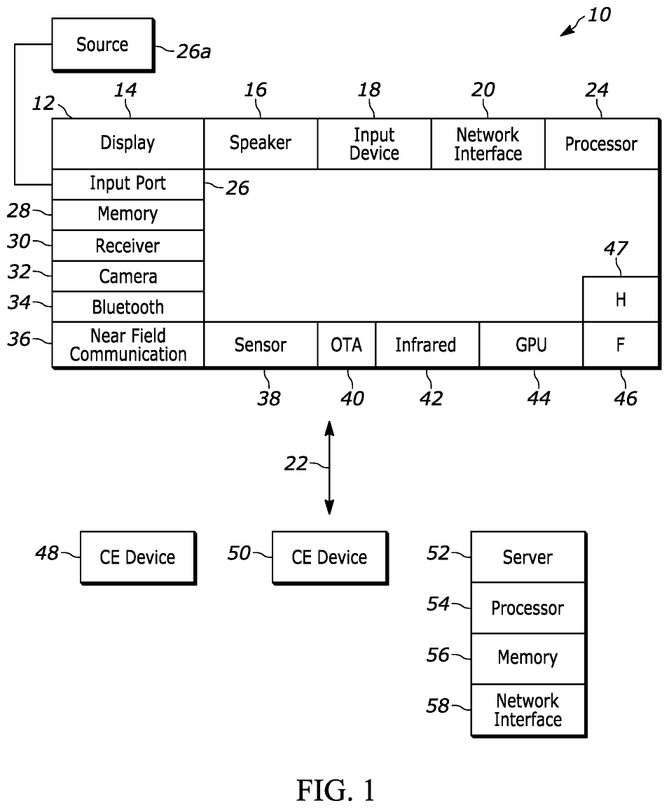

Referring now to FIG. 1, an example system 10 is shown, which may include one or more of the example devices mentioned above and described further below in accordance with present principles. The first of the example devices included in the system 10 is a consumer electronics (CE) device such as an audio video device (AVD) 12 such as but not limited to a theater display system which may be projector-based, or an Internet-enabled TV with a TV tuner (equivalently, set top box controlling a TV). The AVD 12 alternatively may also be a computerized Internet enabled (“smart”) telephone, a tablet computer, a notebook computer, a head-mounted device (HMD) and/or headset such as smart glasses or a VR headset, another wearable computerized device, a computerized Internet-enabled music player, computerized Internet-enabled headphones, a computerized Internet-enabled implantable device such as an implantable skin device, etc. Regardless, it is to be understood that the AVD 12 is configured to undertake present principles (e.g., communicate with other CE devices to undertake present principles, execute the logic described herein, and perform any other functions and/or operations described herein).

Accordingly, to undertake such principles the AVD 12 can be established by some, or all of the components shown. For example, the AVD 12 can include one or more touch-enabled displays 14 that may be implemented by a high definition or ultra-high definition “4K” or higher flat screen. The touch-enabled display(s) 14 may include, for example, a capacitive or resistive touch sensing layer with a grid of electrodes for touch sensing consistent with present principles.

The AVD 12 may also include one or more speakers 16 for outputting audio in accordance with present principles, and at least one additional input device 18 such as an audio receiver/microphone for entering audible commands to the AVD 12 to control the AVD 12. The example AVD 12 may also include one or more network interfaces 20 for communication over at least one network 22 such as the Internet, an WAN, an LAN, etc. under control of one or more processors 24. Thus, the interface 20 may be, without limitation, a Wi-Fi transceiver, which is an example of a wireless computer network interface, such as but not limited to a mesh network transceiver. It is to be understood that the processor 24 controls the AVD 12 to undertake present principles, including the other elements of the AVD 12 described herein such as controlling the display 14 to present images thereon and receiving input therefrom. Furthermore, note the network interface 20 may be a wired or wireless modem or router, or other appropriate interface such as a wireless telephony transceiver, or Wi-Fi transceiver as mentioned above, etc.

In addition to the foregoing, the AVD 12 may also include one or more input and/or output ports 26 such as a high-definition multimedia interface (HDMI) port or a universal serial bus (USB) port to physically connect to another CE device and/or a headphone port to connect headphones to the AVD 12 for presentation of audio from the AVD 12 to a user through the headphones. For example, the input port 26 may be connected via wire or wirelessly to a cable or satellite source 26a of audio video content. Thus, the source 26a may be a separate or integrated set top box, or a satellite receiver. Or the source 26a may be a game console or disk player containing content. The source 26a when implemented as a game console may include some or all of the components described below in relation to the CE device 48.

The AVD 12 may further include one or more computer memories/computer-readable storage media 28 such as disk-based or solid-state storage that are not transitory signals, in some cases embodied in the chassis of the AVD as standalone devices or as a personal video recording device (PVR) or video disk player either internal or external to the chassis of the AVD for playing back AV programs or as removable memory media or the below-described server. Also, in some embodiments, the AVD 12 can include a position or location receiver such as but not limited to a cellphone receiver, GPS receiver and/or altimeter 30 that is configured to receive geographic position information from a satellite or cellphone base station and provide the information to the processor 24 and/or determine an altitude at which the AVD 12 is disposed in conjunction with the processor 24.

Continuing the description of the AVD 12, in some embodiments the AVD 12 may include one or more cameras 32 that may be a thermal imaging camera, a digital camera such as a webcam, an IR sensor, an event-based sensor, and/or a camera integrated into the AVD 12 and controllable by the processor 24 to gather pictures/images and/or video in accordance with present principles. Also included on the AVD 12 may be a Bluetooth® transceiver 34 and other Near Field Communication (NFC) element 36 for communication with other devices using Bluetooth and/or NFC technology, respectively. An example NFC element can be a radio frequency identification (RFID) element.

Further still, the AVD 12 may include one or more auxiliary sensors 38 that provide input to the processor 24. For example, one or more of the auxiliary sensors 38 may include one or more pressure sensors forming a layer of the touch-enabled display 14 itself and may be, without limitation, piezoelectric pressure sensors, capacitive pressure sensors, piezoresistive strain gauges, optical pressure sensors, electromagnetic pressure sensors, etc. Other sensor examples include a pressure sensor, a motion sensor such as an accelerometer, gyroscope, cyclometer, or a magnetic sensor, an infrared (IR) sensor, an optical sensor, a speed and/or cadence sensor, an event-based sensor, a gesture sensor (e.g., for sensing gesture command). The sensor 38 thus may be implemented by one or more motion sensors, such as individual accelerometers, gyroscopes, and magnetometers and/or an inertial measurement unit (IMU) that typically includes a combination of accelerometers, gyroscopes, and magnetometers to determine the location and orientation of the AVD 12 in three dimension or by an event-based sensors such as event detection sensors (EDS). An EDS consistent with the present disclosure provides an output that indicates a change in light intensity sensed by at least one pixel of a light sensing array. For example, if the light sensed by a pixel is decreasing, the output of the EDS may be −1; if it is increasing, the output of the EDS may be a +1. No change in light intensity below a certain threshold may be indicated by an output binary signal of 0.

The AVD 12 may also include an over-the-air TV broadcast port 40 for receiving OTA TV broadcasts providing input to the processor 24. In addition to the foregoing, it is noted that the AVD 12 may also include an infrared (IR) transmitter and/or IR receiver and/or IR transceiver 42 such as an IR data association (IRDA) device. A battery (not shown) may be provided for powering the AVD 12, as may be a kinetic energy harvester that may turn kinetic energy into power to charge the battery and/or power the AVD 12. A graphics processing unit (GPU) 44 and field programmable gated array 46 also may be included. One or more haptics/vibration generators 47 may be provided for generating tactile signals that can be sensed by a person holding or in contact with the device. The haptics generators 47 may thus vibrate all or part of the AVD 12 using an electric motor connected to an off-center and/or off-balanced weight via the motor's rotatable shaft so that the shaft may rotate under control of the motor (which in turn may be controlled by a processor such as the processor 24) to create vibration of various frequencies and/or amplitudes as well as force simulations in various directions.

A light source such as a projector such as an infrared (IR) projector also may be included.

In addition to the AVD 12, the system 10 may include one or more other CE device types. In one example, a first CE device 48 may be a computer game console that can be used to send computer game audio and video to the AVD 12 via commands sent directly to the AVD 12 and/or through the below-described server while a second CE device 50 may include similar components as the first CE device 48. In the example shown, the second CE device 50 may be configured as a computer game controller manipulated by a player or a head-mounted display (HMD) worn by a player. The HMD may include a heads-up transparent or non-transparent display for respectively presenting AR/MR content or VR content (more generally, extended reality (XR) content). The HMD may be configured as a glasses-type display or as a bulkier VR-type display vended by computer game equipment manufacturers.

In the example shown, only two CE devices are shown, it being understood that fewer or greater devices may be used. A device herein may implement some or all of the components shown for the AVD 12. Any of the components shown in the following figures may incorporate some or all of the components shown in the case of the AVD 12.

Now in reference to the afore-mentioned at least one server 52, it includes at least one server processor 54, at least one tangible computer readable storage medium 56 such as disk-based or solid-state storage, and at least one network interface 58 that, under control of the server processor 54, allows for communication with the other illustrated devices over the network 22, and indeed may facilitate communication between servers and client devices in accordance with present principles. Note that the network interface 58 may be, e.g., a wired or wireless modem or router, Wi-Fi transceiver, or other appropriate interface such as, e.g., a wireless telephony transceiver.

Accordingly, in some embodiments the server 52 may be an Internet server or an entire server “farm” and may include and perform “cloud” functions such that the devices of the system 10 may access a “cloud” environment via the server 52 in example embodiments for, e.g., network gaming applications. Or the server 52 may be implemented by one or more game consoles or other computers in the same room as the other devices shown or nearby.

The components shown in the following figures may include some or all components shown herein. Any user interfaces (UI) described herein may be consolidated and/or expanded, and UI elements may be mixed and matched between UIs.

Present principles may employ various machine learning models, including deep learning models. Machine learning models consistent with present principles may use various algorithms trained in ways that include supervised learning, unsupervised learning, semi-supervised learning, reinforcement learning, feature learning, self-learning, and other forms of learning. Examples of such algorithms, which can be implemented by computer circuitry, include one or more neural networks, such as a convolutional neural network (CNN), a recurrent neural network (RNN), and a type of RNN known as a long short-term memory (LSTM) network. Generative pre-trained transformers (GPTT) also may be used. Support vector machines (SVM) and Bayesian networks also may be considered to be examples of machine learning models. In addition to the types of networks set forth above, models herein may be implemented by classifiers.

As understood herein, performing machine learning may therefore involve accessing and then training a model on training data to enable the model to process further data to make inferences. An artificial neural network/artificial intelligence model trained through machine learning may thus include an input layer, an output layer, and multiple hidden layers in between that are configured and weighted to make inferences about an appropriate output.

Indeed, the present assignee has provided techniques for determined human emotion using machine learning. An example of one such technique is set forth in USPP 2023/0372828. These techniques may learn emotion based on one or more of facial expressions, biometrics (e.g., pulse rate, electrochemical sensors, etc.) tone of voice, etc. Present techniques integrate emotion detection technology into the RLHF process, to provide additional input which can be used to evaluate or replace the human's conscious feedback. By capturing video, audio, and/or biometric data in conjunction with the human's evaluation of ML model output, biased responses can be identified and eliminated from training, and misalignment can be detected between conscious and unconscious reaction to content.

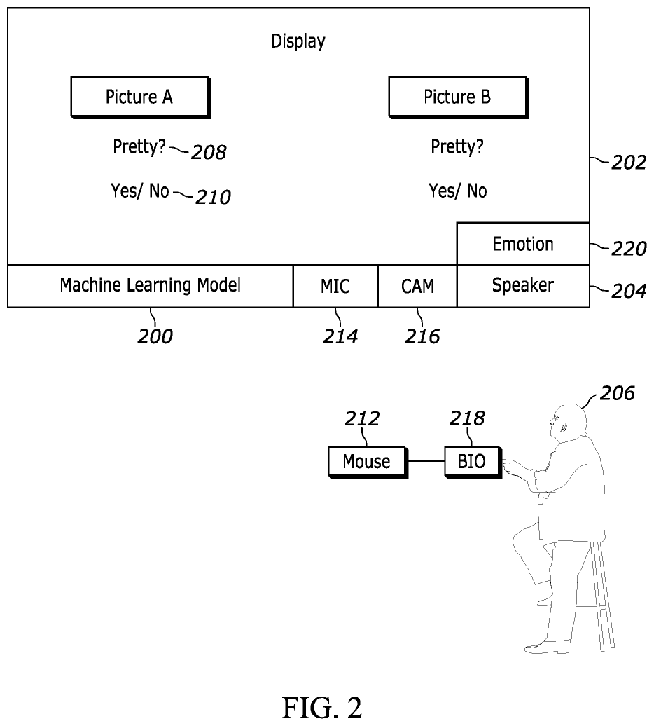

Refer now to FIG. 2. Output from a machine learning (ML) model 200 is output on one or more displays 202 and/or on one or more speakers 204 to be perceived by a tester human 206. The output may be one or more of text or images, and the example output shown in FIG. 2 includes first and second images labeled “picture A” and “picture B”. Thus, the ML model 200 in this example may be a text-to-image generator such as a stable diffusion model that has been trained to generate images from text input, which training is desired to be further refined using RLHF techniques described herein. In other embodiments the ML model may be a generative pre-trained transformer trained to generate lengthier text from a pithy input, which training is desired to be further refined using RLHF techniques described herein. These are two non-limiting examples of ML models that may use present techniques.

As shown in FIG. 2, the person 206 may be presented with a visible and/or audible prompt 208 to indicate whether a particular image is attractive or not. Selectors 210 may be presented for this purpose and may be selected using a manual input device 212 such as a point-and-click device, key entry device, etc. Or, selection may be made by voice as picked up by one or more microphones 214. The input so received is thus a conscious deliberate input.

Additionally, FIG. 2 illustrates that the emotion of the person 206 making the selection may be determined using video of the person as captured by one or more cameras 216, and/or audio from the person as captured by the microphone 214, and/or biometric data as captured by one or more biometric sensors 218 to evaluate the person's emotional state in real time. For example, an emotion detection ML model 220 may be trained on video data to detect when a person is happy, sad, angry, excited, aroused, etc. based on their facial expression. In addition or alternatively the emotion detection ML model 220 may be trained on audio data to detect emotions based on the person's tone of voice, volume, pitch, cadence, word choice, etc. In addition or alternatively the emotion detection ML model 220 may be trained on biometric data as sensed by the biometric sensor 218 such as heart rate, breathing, galvanic skin response, electrical activity in the brain, etc. to correlate specific physical states with emotional states.

For example, the human's facial expressions and/or body language may be captured on video. Their voice may be captured via audio recording as they react verbally to the content. Their biometric data may be captured via devices such as a heart rate monitor, pulse oximeter, EKG machine, fMRI machine, etc.

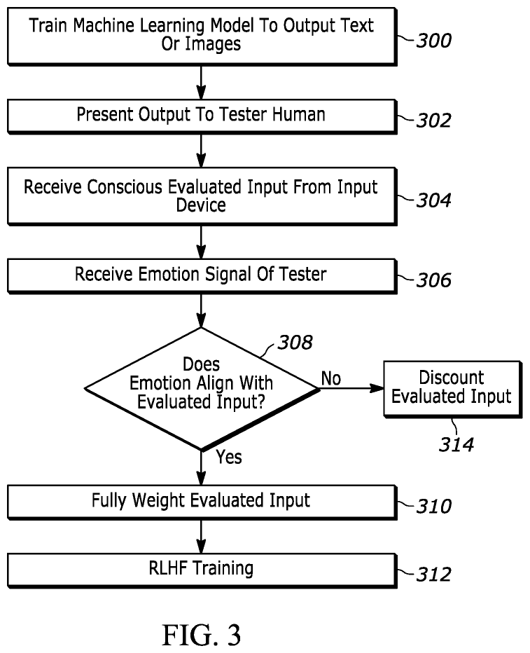

FIG. 3 illustrates that in the context of RLHF, emotion detection technology can be used to monitor the human 206 who is evaluating the ML model 200 output, both during the initial observation of the ML model 200 output (watching the video, seeing the image, hearing the audio, reading the text) as well as during the process of evaluating the output manually.

Commencing at state 300, the ML model 200 shown in FIG. 2 has been pre-trained to output text or images based on input. To execute subsequent RLHF training, output from the ML model 200 is presented at state 302 to a human tester (in practice, many human testers are used). Proceeding to state 304, conscious evaluation input from the tester is received from an input device such as the point and click device 212 in FIG. 2 or as voice command picked up by the microphone 214.

Moving to state 306, emotion signals are received from any of the emotion sensing devices and techniques described herein, indicating the emotion of the person making the input at state 304. Decision diamond 308 indicates that if the emotion aligns with the evaluation input, the evaluation input may be fully weighted at state 310 (in other words, not discounted) and used at state 312 for RLHF training of the model 200. Note that the emotion also may be used.

In determining alignment at state 308, the emotion may be evaluated against the evaluation input to determine how closely the emotion matches the evaluation input as indicated by the emotion satisfying or not a threshold, which may be thought of as an inconsistency threshold. As an example, an emotion indicating dislike or disgust would align with an evaluation input indicating a negative reaction to the output of the ML model 200, whereas such an emotion would not align with an evaluation input indicating a positive reaction and would result in discounting the evaluation input at state 314. As another example, an emotion indicating a rapturous reflexive response to an output of the ML model may only partially align with an evaluation input indicating a mild affinity for the output, in which case, depending on the degree of inconsistency, the evaluation may be fully weighted at state 310 or discounted at state 314. Note that determining alignment may be performed by a ML model trained with ground truth of emotion-evaluation input pairs tagged to indicate whether the pairs are aligned or not and if not, the degree of misalignment.

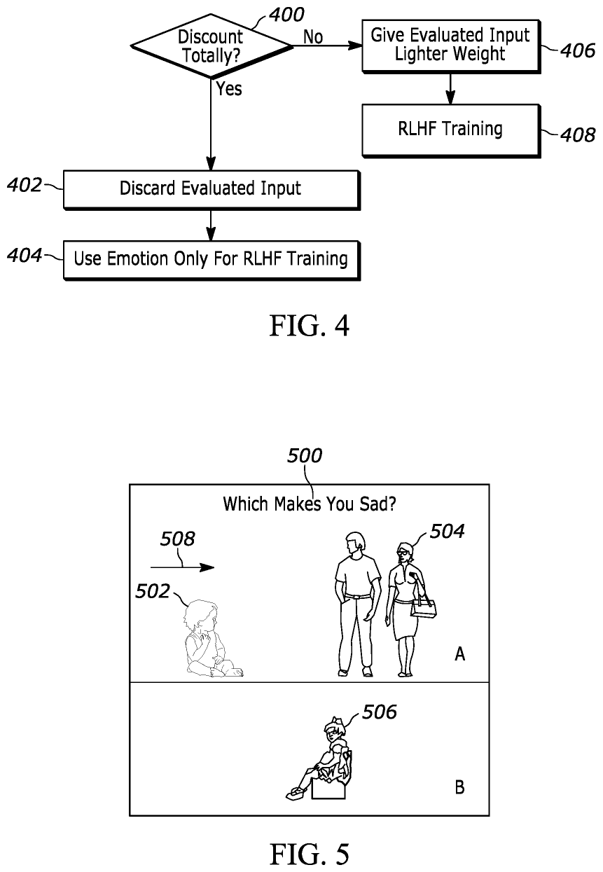

FIG. 4 illustrates discounting further. If it is determined at state 400 that the evaluation input should be completely discounted because the degree of its misalignment with the emotion of the person is extreme, the evaluation input is discarded at state 402. If desired, the emotion and only the emotion may be used at state 404 in RLHF training of the ML model 200 at state 404.

In contrast, if it is not determined at state 400 that the evaluation input should be completely discounted because the degree of its misalignment with the emotion of the person is mild, the logic may move to state 406 to accord the evaluation input a lesser weight than it would otherwise have received at state 310 in FIG. 3, using the partially weighted evaluation input for RLHF training at state 408. The emotion also may be used in this case. It is to be understood that FIG. 4 is intended to be an illustration of an example and is not otherwise limiting unless explicitly so claimed herein.

FIG. 5 illustrates the logic above. In an example, the person 206 is asked to provide feedback on a pair of short video clips generated by the ML model 200 in response to an example text input of “a young boy has lost his parents at the playground.” The person is asked at 500 to evaluate which of the clips made her feel more sad. In Clip A, a toddler 502 is shown crying alone while a group of adults 504 stands nearby. In Clip B, a toddler 506 is shown alone on a swing with a neutral expression, with no other people around.

As the person 206 watches these two clips, her facial expressions may be captured on video by the camera 216 in FIG. 2. In addition, her heart rate, breathing, galvanic skin response, and other biometric signals may be monitored via the sensor 218. This video and biometric data can continue to be captured as the person 206 inputs her evaluation input feedback using, e.g., the point and click device 212 in FIG. 2 and the user interface illustrated in FIG. 5, where she indicates at 508 that Clip A made her feel more sad.

When the person makes her selection of Clip A, additional metadata from the emotion detection system can be appended to her selection. For example, while watching Clip A, the person's facial expression and biometric response may have indicated fear, whereas her emotional response to Clip B may have indicated sadness. Because there is a mismatch between the emotion detected and the conscious response that the person gave, her response may be discounted, scored down, or discarded altogether as described herein.

Alternatively, the data about the person's emotional state can be used directly to train the reward model. If the ultimate goal of the ML model is to generate a video which elicits a specific human emotion, the genuine emotion detected by the system may be more valuable than the conscious feedback the human gives after the fact.

For example, when the person 206 evaluates Clip A versus Clip B, she may knowingly or unknowingly justify to herself reasons why Clip A is more sad. She may reason that the child in Clip A is crying, and that therefore makes Clip A more sad. However, her actual emotional response to Clip A was not sadness but fear, because the presence of other adults that aren't the parents means there is a likelihood that the child may be kidnapped. She may have felt that fear in the moment, but she discounted it upon further reflection and gave a rationalized answer that she felt was more “right.”

On the other hand, the person's genuine emotional reaction to Clip B was actually sadness. The child's expression may have been neutral, but the fact the child was all alone elicited a visceral reaction of sadness. Thus, if it is desired to train the ML model 200 to generate video that can elicit sadness, the person's emotional reaction (as detected via video and biometrics) may be more accurate and valuable than the conscious feedback she provided.

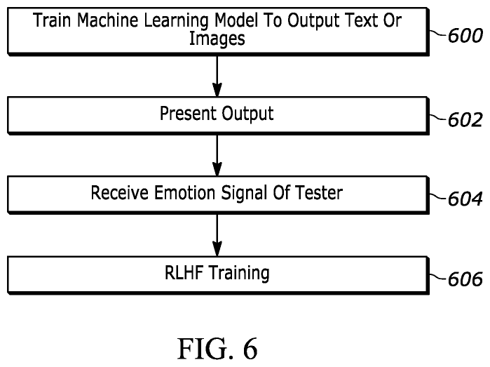

FIG. 6 illustrates an example technique in which reflexive feedback by way of emotion detection is used without conscious evaluation input while a tester human is being shown at least one output of a ML model. FIG. 3 illustrates that in the context of RLHF, emotion detection technology can be used to monitor the human 206 who is evaluating the ML model 200 output, both during the initial observation of the ML model 200 output (watching the video, seeing the image, hearing the audio, reading the text) as well as during the process of evaluating the output manually.

Commencing at state 600, the ML model 200 in FIG. 2 is trained to output text and/or images, in an example. The output is presented at state 602 to a human tester (in practice, many human testers are used).

Proceeding to state 604, emotion signals are received from any of the emotion sensing devices and techniques described herein, indicating the emotion of the tester. The emotion is input to the ML model as RLHF training at state 606.

The technique of FIG. 6 may be can be especially valuable in evaluating entertainment content, where the visceral, real-time reaction to the content can indicate whether it is having the desired effect on the audience.

While the particular embodiments are herein shown and described in detail, it is to be understood that the subject matter which is encompassed by the present invention is limited only by the claims.

What is claimed is:

1. A method comprising:
presenting at least a first output from at least one machine learning (ML) model on at least one display and/or at least one speaker;
receiving a selection with respect to the output;
identifying at least one emotion of a person making the selection; and
responsive to the emotion satisfying an inconsistency threshold with respect to the selection, discounting the selection in training the ML model.

2. The method of claim 1, wherein discounting the selection comprises discarding the selection from training the ML model.

3. The method of claim 1, wherein discounting the selection comprises associating a first weight to the selection in training the ML model, the first weight being less than a second weight of a non-discounted selection.

4. The method of claim 1, comprising receiving the selection via a point-and-click device.

5. The method of claim 1, comprising receiving the selection via a camera.

6. The method of claim 1, comprising receiving the selection via a microphone.

7. The method of claim 1, wherein the output comprises at least one image.

8. The method of claim 1, wherein the output comprises text.

9. The method of claim 1, comprising determining whether the emotion satisfies the inconsistency threshold by comparing the emotion to the selection.

10. A processor system configured to:
associate at least one emotion signal indicating an emotion of a person with at least one evaluation input indicating an evaluation of the person of at least one audio and/or video output of at least one machine learning (ML) model trained to generate text and/or images; and
execute reinforcement learning from human feedback (RLHF) on the ML model according to the association of the emotion signal with the evaluation input.

11. The processor system of claim 10, wherein the output of the ML model comprises text.

12. The processor system of claim 10, wherein the output of the ML model comprises images.

13. The processor system of claim 10, wherein the output comprises audio.

14. The processor system of claim 10, wherein the output comprises at least one image.

15. The processor system of claim 10, wherein the processor system is configured to execute RLHF on the ML model using the evaluation input at a first weight responsive to the association being a first association and execute RLHF on the ML model using the evaluation input at a second weight less than the first weight responsive to the association being a second association.

16. The processor system of claim 15, wherein the second weight is zero such that the evaluation input does not affect RLHF on the ML model.

17. The processor system of claim 10, wherein the processor system is configured to execute RLHF on the ML model using the emotion signal.

18. A device comprising:
a computer memory that is not a transitory signal and that comprises instructions executable by at least one processor system for:
presenting output from a machine learning (ML) model;
receiving at least one emotion signal from at least one person;
correlating the emotion signal with the output; and
executing reinforcement learning on the ML model according to the correlating of the emotion signal with the output.

19. The device of claim 18, wherein the instructions are executable for:
executing reinforcement learning on the ML model according to evaluation input originated by the person.

20. The device of claim 18, wherein the instructions are executable for:
executing reinforcement learning on the ML model according to a relationship between evaluation input originated by the person and the emotion signal.
